(19) United States
(12) Patent Application Publication
(10) Pub. No.: US 2019/0083021 A1
(43) Pub. Date: Mar. 21, 2019

(54) Method, Device And System For Assessing A Subject

(71) Applicant: Appa Scotland Limited, Glasgow (GB)

(72) Inventor: Forbes Hamilton, Glasgow (GB)

(73) Assignee: Appa Scotland Limited, Glasgow (GB)

(21) Appl. No.: 16/133,594

(22) Filed: Sep. 17, 2018

(30) Foreign Application Priority Data
Sep. 18, 2017 (GB) 1715012.9

(51) Int. Cl. A61B 5/16; G06K 9/00; G06F 21/44

(52) U.S. Cl. CPC A61B 5/164; A61B 5/165; A61B 5/024; G06F 21/44; G06K 2209/01; G06K 9/00288

(57) ABSTRACT

There is provided a method, device and system for performing an assessment of a subject, in particular a human subject, based on the detection of physical responses to stimuli. The method includes: providing a reference signature of the subject; obtaining a measurement signature corresponding to at least one physical response to a stimulus; comparing the measurement signature to the reference signature; and generating an output signal in response to the comparison; wherein the reference signature and the measurement signature conform to a predetermined format.

### TECHNICAL FIELD

The present disclosure relates to a method, device and system for assessing a subject. More particularly, the present disclosure relates to a method of assessing a human subject based on the detection of physical responses to stimuli, a device for executing said method, and a system for executing said method.

### BACKGROUND ART

The detection of deception and mental health signs has historically been heavily based on subjective observation and interpretation. Training and skills required to appropriately assess a subject are extensive, and even when available, can often lack consistency and accuracy. Assessments of a subject can typically take a long period of time, making it impractical to rely solely on a human agent to assess the mental health of a subject.

Whilst research efforts have been applied to developing a more objective approach to the detection of deception, the methods used for such a purpose typically rely on an expensive set-up and generally require a high level of skill to operate and to interpret results.

There is therefore a need to develop a portable, automated system of assessing the mental health of a subject, for such purposes as detecting deception or an adverse emotional response. The use of legacy devices in such a system would increase the accessibility to the methods developed herein without compromising general performance of the device. There is a further need to enable the assessment of a plurality of users substantially simultaneously, in order to reduce delays in waiting for an assessment.

The assessment of a subject's mental health is heavily dependent on the subject itself. Indicators that are known to be reliable for one individual may not be reliable for another. There is therefore a need to develop a system for assessing the mental health of a subject that is customised to the individual subject.

### Statement of Invention

An aspect of the present disclosure is to provide a method for performing an assessment of a subject. The method comprises providing a reference signature of the subject, obtaining a measurement signature corresponding to at least one physical response to a stimulus, comparing the measurement signature to the reference signature, and generating an output signal in response to the comparison. The reference signature and the measurement signature conform to a predetermined format.

This method enables the detection of physical responses that are associated with stress or dishonesty. The reference signature is used as a baseline with which to compare the measurement signature. As the format of both signatures is known, it is possible to compare the known baseline physical responses to the measured physical responses. Knowledge of the format enables mapping between characters in the signature to an associated physical response. The method may be applied to a large number of subjects, at arbitrary times, with enhanced accuracy and consistency over routine assessment by a human agent (i.e. mental health professional, therapist, etc.).

A second aspect of the present disclosure is to provide an electronic device, comprising at least one sensor configured to detect at least one physical response, a communication unit configured to transmit and receive data, and a processor configured to control output of a stimulus to a subject, generate a measurement signature based on the detected at least one physical response, and generate an output signal. The measurement signature conforms to a predetermined format.

The electronic device performs at least some of the operations of the method. This enables the adaptation of the present disclosure on a variety of devices with potentially different hardware and software configurations.

A third aspect of the present disclosure is to provide a server, comprising a memory configured to store a reference signature, an interface unit configured to receive a measurement signature and to transmit an output signal, a processing unit configured to perform a comparison between the received measurement signature and the stored reference signature and to generate the output signal based on the comparison. The reference signature and the measurement signature conform to a predetermined format.

The use of a server to perform the comparison may allow a plurality of comparisons to take place simultaneously. Moreover, performing the comparison between the reference signature and the measurement signature may require more processing power than is available on the electronic device. Use of the server to perform the comparison therefore enables normal operation of the electronic device to continue with little or no interruption. The method could therefore be deployed on a variety of electronic devices and servers with varying available resources.

### BRIEF DESCRIPTION OF THE DRAWINGS

These and other features will be made apparent with reference to the description, the appended claims, and the accompanying figures.

FIG. 1 is an overview of a system implementing an electronic device and a server.

FIG. 2 is a block diagram illustrating the components of an electronic device and a server.

FIG. 3 is a flowchart illustrating a method of assessing a subject.

FIG. 4 is a flowchart illustrating a method of generating a signature.

FIG. 5 is a diagram indicating the interoperation of an electronic device and a server.

FIG. 6 is a diagram indicating the interoperation of an electronic device, a server and an external device.

FIG. 7 is a diagram indicating a method of assessment where authentication is used.

FIG. 8 is a diagram indicating a method of updating a reference signature.

FIG. 9 is an illustration of a graphical user interface displayed on an electronic device with which the subject may interact.

### DETAILED DESCRIPTION OF THE DRAWINGS

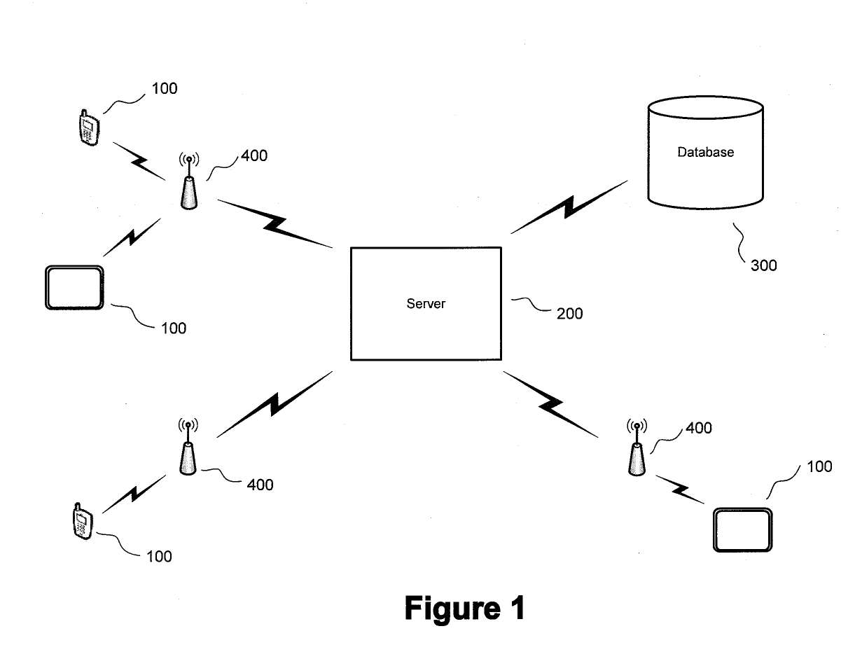

FIG. 1 is an overview of a system implementing an electronic device and a server.

Referring to FIG. 1, the system 10 may include at least one electronic device 100, a server 200, and, optionally, a database 300 and a network access point 400.

The electronic device 100 may be a mobile electronic device, such as a mobile phone, smartphone, personal digital assistant (PDA), tablet PC, notebook PC, laptop, ultrabook, wearable device, appcessory, desktop computer, smart home appliance or the like. The electronic device 100 may be configured to access a wireless and/or wired network (for example: a WiFi network, a wide area network (WAN) such as a cellular telecommunication network, an xDSL network, and/or an Ethernet network).

In some embodiments, the method described herein may be performed entirely by the electronic device 100, or may be performed in part by the electronic device and in part by another electronic device and/or the server 200.

In some embodiments, the database may be stored in the electronic device 100. The database may additionally, or alternatively, be stored in the memory of the server 200.

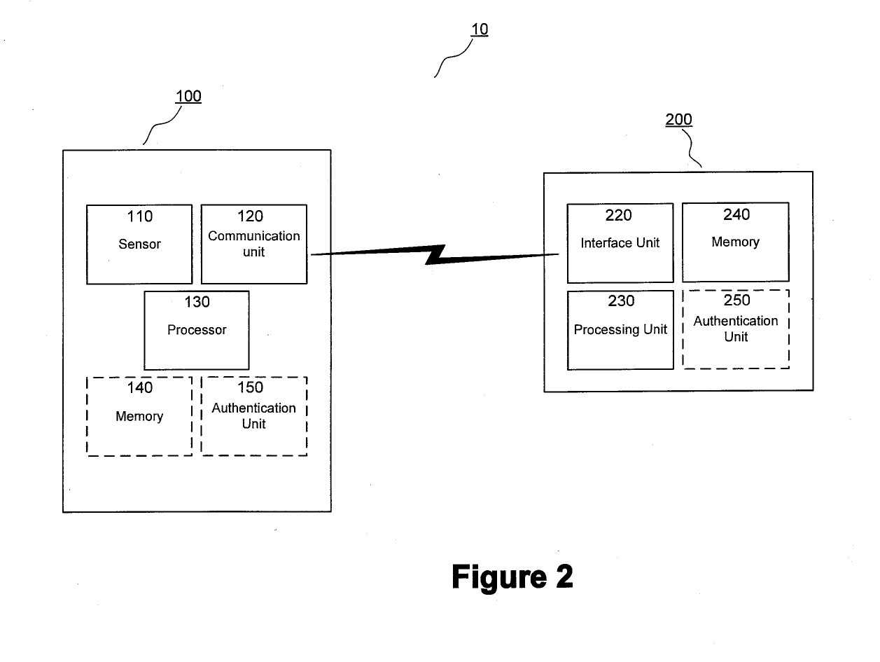

FIG. 2 is a block diagram illustrating the components of an electronic device and a server.

Referring to FIG. 2, an electronic device 100 may comprise at least one sensor 110, a communication unit 120 and a processor 130. In some embodiments, the electronic device 100 may further comprise a memory 140 and/or an authentication unit 150.

The at least one sensor 110 may embedded in the electronic device 100, such as a camera (i.e. a digital camera arranged to capture still images and/or video images), a microphone, a timer, or the like. The at least one sensor 110 may alternatively be communicatively coupled to the electronic device 100. The at least one sensor 110 may measure time, colour, pitch, volume, movement, or the like. The at least one sensor 110 may be configured to perform character recognition, facial recognition, or the like.

The communication unit 120 may be configured to transmit and/or receive data from an external device, such as server 200 or another electronic device.

The processor 130 may be configured to receive information from the at least one sensor 110. The processor 130 may then be configured to assign value(s) to characteristics measured by the at least one sensor 110. The processor 130 may then be configured to combine or arrange the assigned value(s) to create a signature.

Signatures are used herein to enable a comparison of physical responses invoked by a measurement stimulus with baseline physical responses invoked by at least one baseline stimulus.

A reference signature is generated when a user initialises an account. A series of stimuli are output by the electronic device, and characteristics of the response to each stimulus are detected and assessed. For example, physical responses such as heart rate, breathing rate, time delay in responding to the stimulus, eye gaze, and the like. Such physical responses are generally associated with stress and are used to indicate dishonesty.

The reference signature is used as a baseline to determine how the subject responds to an assessment.

In some embodiments, in order to allow comparison, the reference signature and the measurement signature may conform to the same predetermined signature template.

The signature template may be a data structure comprising one or more fields. Each field of the signature template may be of a predetermined size: the predetermined size may be expressed as a size of the data representation, such as a number of Unicode, alphanumeric or binary characters. Each field of characters may relate to one physical response that is expected to be measured by a sensor. The fields may be arranged in a predetermined order. Each data representation of a given field of the reference signature and the measurement signature conforming to the template may have a required format, such as an alphanumeric value, a bit (i.e. 1 or 0), a numeric value, or an alphabetic value. There may be a range of acceptable values for each character and/or field. The fields may all have the same format requirements, or each field may have a required format.
Example Signatures:
Template 1: e.g. 110010110
Size: 9 characters
Format: Binary
Mapping:
Index—corresponding physical response
   [0]— eye gaze: if averted, =1, otherwise 0
   [1]— response delay: if greater than 0.5 ms, =1, otherwise 0
   [2]— blushing: if colour change greater than threshold, =1, otherwise 0 etc.
Template 2: e.g. abbaced
Size: 7 characters
Format: alphabetic
Mapping:
Index—corresponding physical response
   [0]—eye gaze: if averted, =b, otherwise a
   [1]—response delay: if greater than 0.5 ms, =c, if less than 0.5 ms but greater than 0.25 ms, =b, otherwise a
   [2]—blushing: if colour change greater than first threshold, =c, if colour change lower than first threshold but greater than second threshold, =b, otherwise a etc.
Template 3: e.g. blce0d35
Size: 8 characters
Format: Alphanumeric
Mapping:
Index—corresponding physical response
   [0]—response delay: if greater than 0.5 ms, =c, if less than 0.5 ms but greater than 0.25 ms, =b, otherwise a
   [1]—eye gaze: if averted, =1, otherwise 0
   [2]—blushing: if colour change greater than first threshold, =c, if colour change lower than first threshold but greater than second threshold, =b, otherwise a etc.

In the example of Template 3, different characters have different formats. Some physical responses are measured using different values—some may be binary, whilst others may have more than 2 potential values.

Signatures conforming to the signature template would thus have the same size, such as number of characters, with values falling into the appropriate range. Knowledge of the signature template therefore allows suitable interpretation of the characters in the signature, and appropriate comparisons to be made.

The signature template may be selected from a plurality of available templates, based on the type of device used and the sensors available.

The signature template maps the order of characters in the signature to the appropriate physical responses, enabling a character-to-character comparison between signatures conforming to the same signature template. This ensures that values relating to the same physical response(s) are being compared.

The electronic device 100 may further comprise a display (not shown). The display may output a stimulus to a user in the form of text.

The electronic device 100 may further comprise a speaker (not shown). The speaker may output a stimulus to a user in the form of an audio signal.

In some embodiments, the electronic device 100 may comprise both a display and a speaker. A stimulus may be output by the display in the form of text and, simultaneously, by the speaker in the form of an audio signal.

A stimulus is an output that prompts some form of physical response from a subject. In some embodiments, stimuli may be expressed as questions, either written, verbal, or both written and verbal. The stimuli may also be expressed in the form of a written or verbal statement, a video clip, an audio clip, an image, or the like. A stimulus is generally selected to elicit a reaction from the subject, which can be measured through the use of at least one sensor.

The subject may be a human subject, an animal subject, and artificial intelligence (AI) subject or a digital subject, but is not limited thereto.

In some embodiments, the mental health of a human subject may be assessed. In particular, the honesty level or stress level of a subject may be assessed.

The subject may respond to a stimulus in one of several manners, including, but not limited to, a verbal response, a typed or written response, or a body-language based response.

There are several categories of stimuli that may be used. Reference stimuli are used in the generation or establishment of a reference signature. The reference stimuli are selected such that emotive responses are less probable. Higher brain functions are not typically engaged when responding to reference stimuli.

Measurement stimuli are those to which a stronger reaction is more likely to occur. Whilst the answer provided by the subject is of little relevance, the physical reaction is considered significant. Upon output of a measurement stimulus, the subject's physical response is detected and assessed through the use of at least one sensor. More than one physical response may be detected. Any responses detected by the at least one sensor may be used in the generation of a measurement signature.

Based on a measured response to an initial measurement stimulus, a subsequent stimulus from a third category of stimuli may be directed to the subject. The third category comprises measurement stimuli that relate to an initial measurement stimulus, but are typically more specific in nature than the initial measurement stimulus. In other words, an initial measurement stimulus may indicate some level of stress using a broad question, and further information can then be obtained by exploring finer details of the broad questions. Questions narrower in scope can be selected as subsequent stimuli, for example.

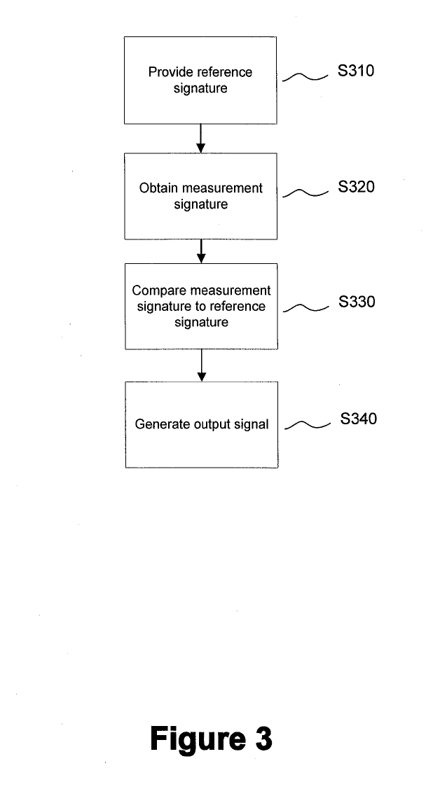

FIG. 3 is a flowchart illustrating a method of assessing a subject.

Referring to FIG. 3, at operation S310, a reference signature is provided to the electronic device 100. The reference signature may be generated by the electronic device 100, stored locally in memory 140 of the electronic device 100, or stored remotely in memory 240 of the server 200. The reference signature may be received by the electronic device 100 from an external device, such as server 200, or another electronic device.

In operation S320, a measurement signature is obtained. The measurement signature may be generated by the electronic device 100 through the use of at least one sensor 110 embedded or connectively coupled to the electronic device 100. The measurement signature may be generated by an external device, such as server 200 or another electronic device, and transmitted to the electronic device 100.

In operation S330, the measurement signature and the reference signature are compared. The comparison may be performed character-wise, where the first character of the measurement signature is compared to the first character of the reference signature, the second character of the measurement signature is compared to the second character of the reference signature, and so forth. In some embodiments, it may be sufficient to compare a subset of the characters in each signature. For example, after comparing the first N characters in the measurement signature to the corresponding first N characters in the reference signature, it may be determined that there is already a significant difference or deviation from the reference signature, and comparison of further characters may not be required.

In some embodiments, the characters may have an associated weighting, determined by their position in the signature or otherwise indicated in the template. In other embodiments, all characters may be equally weighted.

Operation S330 may comprise establishing a degree of difference between the measurement signature and the reference signature. In such an embodiment, the degree of difference may need to exceed a threshold, used to indicate whether a detected difference is deemed significant. The threshold may be set to a predetermined value and/or may have a value determined by statistical, empirical and/or experimental means. A difference exceeding said threshold would be considered sufficiently different, and a stressed or dishonest reaction to a stimulus may be detected. A difference less than said threshold may be considered to be an insignificant fluctuation, or may not merit further probing. Further thresholds may be applied to allow for finer discrimination of degrees of significance.

Operation S330 may be performed by the server 200. In other embodiments, operation S330 may be performed by the electronic device 100. By performing the comparison operation on the server 200, the resource requirements for the electronic device 100 can be reduced. This enables the method to be accessible to a range of electronic devices with varying available resources (both hardware and software). Reducing the required resources on the electronic device 100 may also enable the method to be performed in the background.

In operation S340, an output signal is generated. In some embodiments, the server 200 generates the output signal, which is subsequently transmitted to the electronic device 100 and/or an external device. The output signal may be used to select a subsequent stimulus from a plurality of stimuli. In some embodiments, the output signal may be used to send a message (such as a warning message) to an external device. In some embodiments, the output signal may be used to allow or deny access to a device, an account, a room or building, and so forth. In some embodiments, the output signal may be used to signal the completion of the comparison operation. The output signal may comprise the result of the comparison operation.

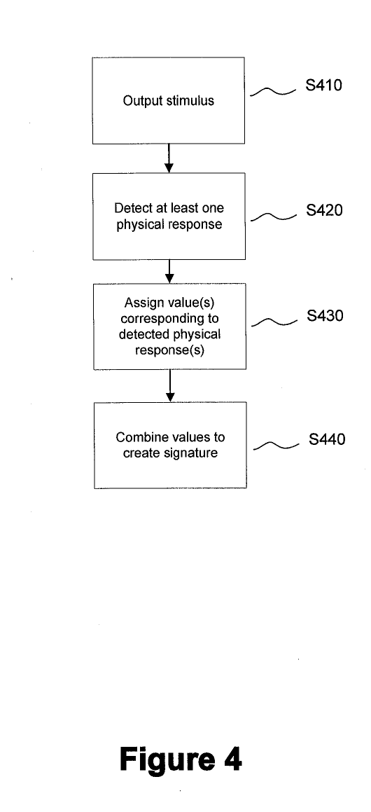

FIG. 4 is a flowchart illustrating a method of generating a signature.

There are two types of signatures that may be generated according to the method described with reference to FIG. 4—a reference signature and a measurement signature. The signature generation method for generating a reference signature is similar to the signal generation method for generating a measurement signature, with the most significant difference pertaining to the choice of stimulus. In generating a reference signature, reference stimuli are used. As described previously, a reference stimulus is selected such that higher brain functions are not engaged. Reference stimuli are designed such that they do not elicit a complex emotive response. In generating a measurement signature, however, a measurement stimulus is used. A measurement stimulus is specifically crafted to elicit an emotive response, or a response that requires the use of higher brain functions. A response to a stimulus generally extends beyond the answer provided by the user, but rather focuses on the body language and other, generally subconscious, characteristics that are used in detecting dishonesty or stress. Such responses typically result in physical manifestations.

Referring now to FIG. 4, in operation S410, a stimulus is output. The choice of stimulus is dependent on the type of signature being generated. If a reference signature is being generated, then the stimulus will be selected from a plurality of reference stimuli. Conversely, if a measurement stimulus is being generated, then the stimulus will be selected from a plurality of measurement stimuli.

The stimulus may be output on a display of the electronic device 100 in a visual form. The stimulus may be textual, or it may be displayed in an image form. The stimulus may additionally or alternatively be output audibly, via a speaker communicatively coupled or embedded in the electronic device 100. The stimulus may additionally or alternatively take the form of a tactile output, such as a vibration or series of vibrations.

In operation S420, at least one physical response is detected by at least one sensor 110 embedded or communicatively coupled to the electronic device 100. The at least one physical response may be a delay responding to the stimulus. For example, if the stimulus is a question delivered audibly or visually or both, then a timer can detect a delay between the conclusion of the stimulus and the input of an answer, which may be detected via a microphone and/or a text input or touchscreen input.

The at least one physical response may be related to eye gaze, fluctuations in facial colouring (e.g. blushing), pupil dilation, sweating, changes in heart rate and/or breathing rate, changes in voice pitch, faltering, stammering, stuttering, twitching, changes in blinking, or other generally inadvertent physical characteristics.

In operation S430, at least one value is assigned to each detected physical response. As described previously, the template determines the order in which fields of characters corresponding to each physical response appear in the signature. The at least one value may be one or more binary value (either 0 or 1), numeric character (for example, an integer), alphabetic character, or alphanumeric character. According to the predetermined template, the at least one value may have a range of acceptable values. In some embodiments, not all of the physical responses designated in the template are measured—in these cases, a placeholder character may be inserted accordingly, in order to prompt the comparison operation to ‘skip’ the comparison of values corresponding to the indicated physical response(s).

In operation S440, the at least one value for each detected physical response are arranged or combined to create a signature. The values may be concatenated according to a predetermined order in accordance with the template. Additionally or alternatively, at least some values may be combined using known statistical and/or mathematical methods.

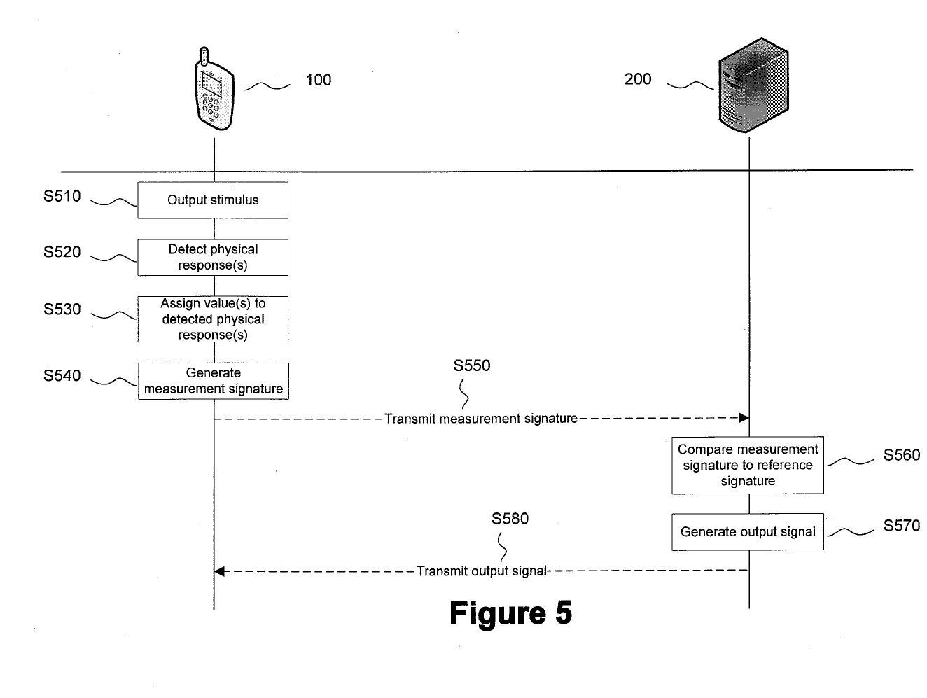

FIG. 5 is a diagram indicating the interoperation of an electronic device and a server. Referring to FIG. 5, an electronic device 100 and a server 200 are provided.

Operations S510, S520, S530 and S540 correspond to operations S410, S420, S430 and S440 respectively, which have been described in reference to FIG. 4. For the purposes of brevity, these operations will not be described in detail.

In operation S550, the electronic device 100 transmits the measurement signature generated in operation S540 to the server 200. Upon receiving the measurement signature, the server 200 compares the measurement signature to the reference signature corresponding to the subject being assessed, in operation S560. The comparison between the reference signature and the measurement signature has been described in detail in reference to operation S330 of FIG. 3, and will therefore not be repeated here in detail.

In operation S570, based on the result of the comparison between the measurement signature and the reference signature obtained in operation S560, the server 200 generates an output signal. This operation has been described in reference to operation S340 of FIG. 3, and will not be repeated here.

In operation S580, the server 200 transmits the output signal to the electronic device 100.

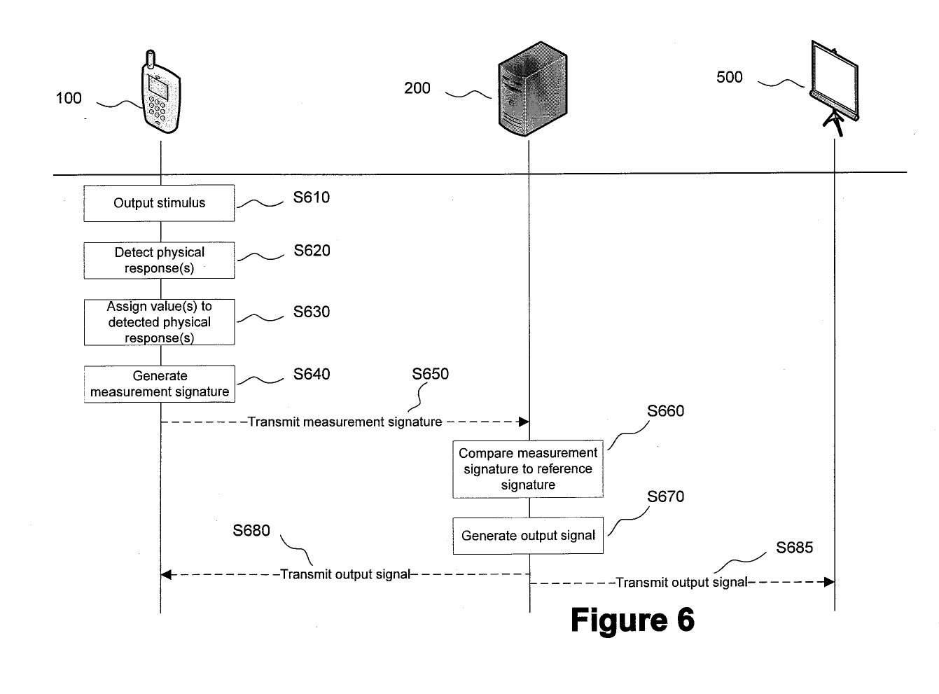

FIG. 6 is a diagram indicating the interoperation of an electronic device, a server and an external device.

Referring to FIG. 6, an electronic device 100, a server 200 and an external device 500 perform operations in a method of assessing a subject.

Operations S610, S620, S630, S640, S650 and S660 correspond to operations S510, S520, S530, S540, S550 and S560 of FIG. 5. A detailed description of these operations is therefore omitted.

In operation S670, the server 200 generates at least one output signal based on the comparison between the measurement signature and the reference signature performed in operation S660. In some embodiments, the server 200 generates a single output signal. In other embodiments, the server 200 may generate a plurality of output signals. One of the output signals may be generated for the purpose of being transmitted to the electronic device 100. A distinct output signal may also be generated for the purpose of being transmitted to the external device 500. In some embodiments, these output signals may be the same. In some embodiments, one of the output signals may entirely comprise the other. For example, the output signal transmitted to the external device 500 in operation S685 may comprise the output signal that is transmitted to the electronic device 100 as well as some additional information. In another example, the output signal generated for transfer to the electronic device 100 may comprise the output signature generated for transfer to the external device 500, as well as some additional information. In another example, the output signal generated for transfer to the electronic device 100 and the output signal generated for transfer to the external device 500 may comprise common information as well as some specific information.

The output signal intended for the electronic device 100 may, for example, comprise information regarding the selection of a next stimulus. The output signal intended for the external device 500 may comprise information such as an alert to a supervisor or a trigger to disable access to an account, a room or building, or the like.

In operation S680, the server 200 transmits the output signal intended for the electronic device 100 to the electronic device 100.

In operation S685, the server 200 transmits the output signal intended for the external device 500 to the external device 500.

Operations S680 and S685 may take place simultaneously, or one may precede the other.

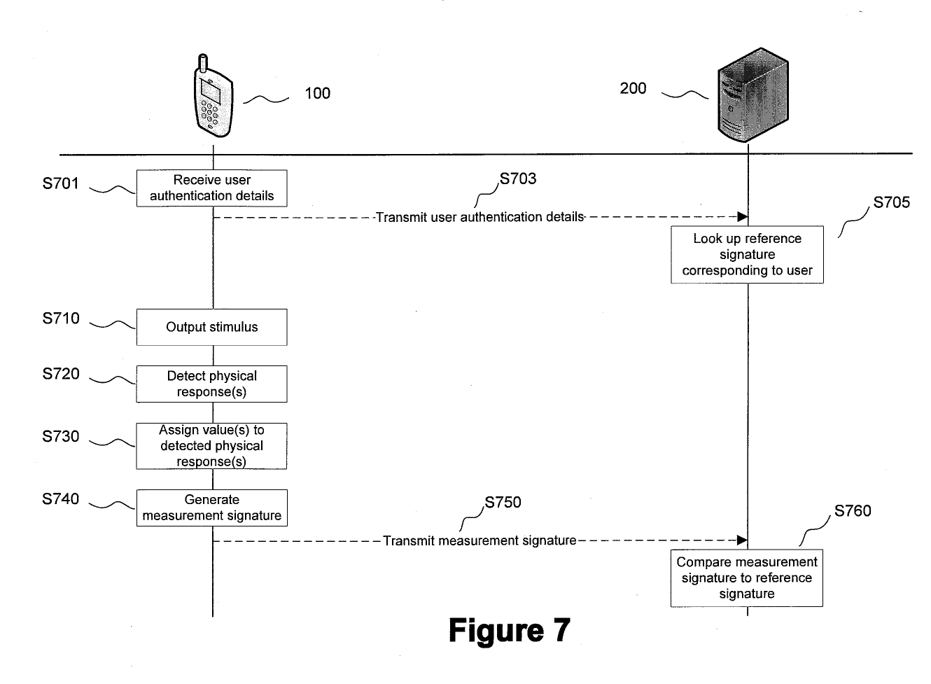

FIG. 7 is a diagram indicating a method of assessment where authentication is used.

Referring now to FIG. 7, electronic device 100 and server 200 perform operations to authenticate a user prior to performing the method illustrated, for example, in FIG. 5 or 6.

In operation S701, the electronic device 100 receives user authentication details. The user authentication details may be input by the user into the electronic device 100, or may be automatically determined. In some embodiments, a password, passcode, written signature, or the like may be input. In some embodiments, voice recognition, fingerprint recognition, facial recognition or the like may be used for authentication. In some embodiments, the electronic device 100 is authenticated, for example, by means of a public and/or private key. The electronic device 100 may be associated with a user. In some embodiments, a combination of multiple authentication details may be required. In some embodiments, there may be a limit to the number of incorrect authentication attempts made, or there may be a threshold amount of time within which to enter an accepted authentication. In some embodiments, the authentication unit 150 of the electronic device 100 is configured to authenticate the user based on the authentication details provided. In some embodiments, the authentication is performed by the authentication unit 250 of the server 200 based on the authentication details provided.

The authentication details may comprise a user identification value, such as a username or a user ID. In some embodiments, the user identification value is anonymised. In operation S703, the authentication details are transmitted by the electronic device 100 to the server 200. The transmission of the authentication details may be triggered automatically upon input, or may be sent upon a manually input request to transmit the authentication details, for example, as a “Log In” button. The authentication details may further comprise identification information of the electronic device 100, which may indicate the capabilities of the electronic device 100.

Upon receiving the user authentication details from the electronic device 100, the server 200 determines user identification information from the user authentication details. In operation S705, the user identification information is used by the server 200 to identify a reference signature corresponding to the user. The reference signature may be stored in a database in the memory 240 of the server 200.

The memory 20 of the server 200 may comprise a plurality of reference signatures corresponding to the user. The plurality of reference signatures may conform to a plurality of signature templates. The electronic device 100 and/or the associated at least one sensor 110 may be used to select the most relevant, or the most recent, reference signature. For example, a first electronic device may comprise a first set of sensors capable of detecting and measuring a first set of physical responses. A second electronic device may be coupled to a different set of sensors capable of detecting and measuring a different set of physical responses. Therefore, based on the capabilities of the electronic device 100, the most relevant reference signature may be selected.

Either meanwhile or subsequently, the electronic device 100 outputs a stimulus in operation S710. Operations S710, S720, S730, S740, S750 and S760 have been described in detail with reference to FIGS. 3 to 6. As such, a detailed description will be omitted here.

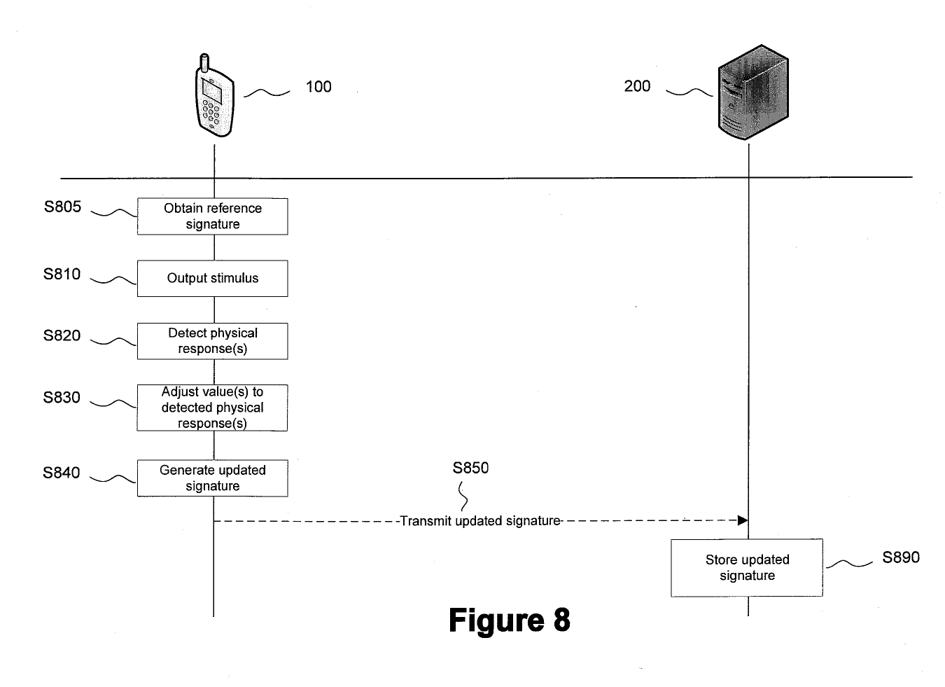

FIG. 8 is a diagram indicating a method of updating a reference signature.

Referring now to FIG. 8, electronic device 100 and server 200 may be configured to update or refine a reference signature. In operation S805, the electronic device 100 obtains the reference signature. This is described in detail with reference to operation S310 of FIG. 3, and as such, a detailed description of operation S805 will be omitted. In some embodiments, the reference signature may be used to determine which physical responses are to be measured. In some embodiments, the reference signature may be updated by changing the signature template and adjusting the amount of information (for example, the number of characters or their allowed range of values) incorporated in the reference signature.

Operations S810 and S820 correspond to operations S510 and S520 of FIG. 5. For the sake of brevity, these operations will not be described in detail here.

In operation S830, the values contained within the reference signature may be adjusted. By detecting physical responses to similar stimuli repeatedly, the accuracy of the reference signature may be improved. When a physical response is detected several times, a mean value, or other statistical quantity, may be calculated. As the detection of a physical response is repeated—for example, in response to a plurality of reference stimuli—the associated error decreases. The error, such as a standard error, may be used in determining threshold values that are customised to a particular user. For example, the change in forehead colouring may have only a small fluctuation with one user, and even a slight change in colouring may be indicative of an increased stress level or a decreased honesty level. In another user, the forehead colouring may have a much larger natural fluctuation, and a much higher threshold would be required to determine if it is indicative of an increased stress level or a decreased honesty level.

Operations S810 to S830 may be repeated. In each repetition, a different stimulus is output. As the number of iterations of these operations increases, the errors corresponding to the detected physical responses may be seen to decrease, and the accuracy of the reference signature may be improved.

In operation S840, the reference signature may be updated with the adjusted values. The associated errors relating to each physical response may also be updated.

In operation S850, the updated signature is transmitted to the server 200. In some embodiments, the updated signature may be transmitted with additional statistical information, such as error information or an updated range of accepted values.

Upon receiving the updated signature, the server 200 stores the updated signature in its memory 240. The updated signature may be stored in a database, either in the server 200 or externally. The updated signature may override an existing signature, or may be stored in a version-controlled system. Additional information that may be received with the updated signature, such as statistical information, may also be stored in the memory 240 or in a database.

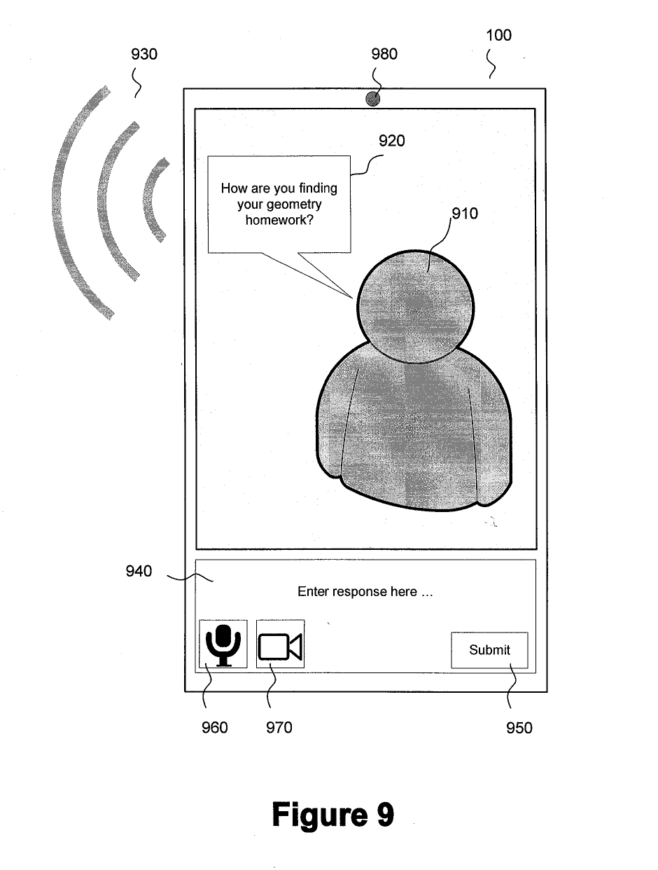

FIG. 9 is an illustration of a graphical user interface displayed on an electronic device with which the subject may interact.

Referring to FIG. 9, an electronic device 100 comprising a display 900, a speaker 930, a camera 980, and/or an input region 940, may be used to output an avatar 910, a visual stimulus 920 and/or an audible stimulus 930. The graphical user interface displayed on the electronic device 100 may comprise a microphone input button 960, a camera input button 970 and/or a submit button 950.

The electronic device 100 may comprise a display 900, upon which the graphical user interface may be displayed. The graphical user interface may comprise an avatar 910, which speaks, engages eye contact and elicits a reaction. The use of said avatar 910 engages the emotional centre of the brain, which generates a subconscious response originating in the limbic system: specifically the amygdala for emotional response and thalamus (motor function) and cerebral cortex (emotional response and motor function). The graphical user interface may comprise an engine with multi-sensory impact, which engages higher brain function to a greater level than would be engaged in a simple paper or screen questionnaire.

The graphical user interface may output a visual stimulus, such as the question posed by the avatar in speech bubble 920, an audible stimulus, such as an audio version of the question output by a speaker, as illustrated by reference sign 930, or both may occur simultaneously.

The user (also referred to as the subject) may enter a response in the input region 940, either by typing, writing, drawing, or the like. The input region 940 also comprises several buttons for various forms of input, such as a microphone button 960, which enables a speech-based input, and/or a camera button 970, which enables a camera-based input, such as an image or video. In some embodiments, if a camera-based input is selected, the microphone function of the electronic device 100 may be simultaneously automatically triggered. If the camera-based input is selected, camera 980 may be used to capture a video or image of the user.

In some embodiments, a ‘Submit’ button 950 may be used to indicate that a text-based or written response is ready for submission. In some embodiments, the text-based or written input is submitted automatically, without the need to press the ‘Submit’ button 950.

1. A method for performing an assessment of a subject, the method comprising:
providing a reference signature of the subject;
obtaining a measurement signature corresponding to at least one physical response to a stimulus;
comparing the measurement signature to the reference signature; and
generating an output signal in response to the comparison;
wherein the reference signature and the measurement signature conform to a predetermined format.

2. The method of claim 1, wherein the predetermined format comprises a template for a plurality of characters arranged in a predetermined order.

3. The method of claim 1, wherein obtaining the measurement signature comprises generating the measurement signature, by:
outputting, by an electronic device, the stimulus to the subject;
detecting, by at least one sensor coupled to the electronic device, the at least one physical response to the stimulus;
assigning at least one measurement value corresponding respectively to each of the detected at least one measurement physical response; and
combining the assigned measurement values in the predetermined order.

4. The method of claim 1, wherein providing the reference signature comprises receiving the reference signature from an external device.

5. The method of claim 1, wherein providing the reference signature comprises generating the reference signature.

6. The method of claim 5, wherein generating the reference signature comprises providing, by an electronic device, a first baseline stimulus to the subject;
detecting, by at least one sensor coupled to the electronic device, at least one baseline physical response to the first baseline stimulus;
assigning at least one value corresponding respectively to each of the detected at least one baseline physical response;
combining the assigned values in accordance with the predetermined format of the reference signature corresponding to the detected at least one baseline physical response.

7. The method of claim 1, further comprising:
providing a second baseline stimulus to the subject;
detecting, by the at least one sensor coupled to the electronic device, at least one updated baseline physical response;
assigning at least one updated value in the predetermined order corresponding to the detected at least one updated baseline physical response;
updating the baseline signature based on at least one updated assigned value.

8. The method of claim 7, further comprising transmitting the updated baseline signature to an external device.

9. An electronic device, comprising:
at least one sensor configured to detect at least one physical response;
a communication unit configured to transmit and receive data; and
a processor configured to:
control output of a stimulus to a subject;
generate a measurement signature based on the detected at least one physical response; and
generate an output signal;
wherein the measurement signature conforms to a predetermined format.

10. The electronic device of claim 9, wherein the predetermined format comprises a template for a plurality of characters arranged in a predetermined order.

11. The electronic device of claim 9, further comprising a memory configured to store a reference signature, the reference signature conforming to the predetermined format.

12. The electronic device of claim 11, wherein the communication unit is further configured to receive a reference signature, the reference signature conforming to the predetermined format.

13. The electronic device of claim 9, wherein the processor is further configured to generate a reference signature, the reference signature conforming to the predetermined format.

14. The electronic device of claim 9, wherein the communication unit is further configured to transmit the measurement signature to an external device.

15. A server, comprising:
a memory configured to store a reference signature;
an interface unit configured to:
receive a measurement signature; and
transmit an output signal;
a processing unit configured to:
perform a comparison between the received measurement signature and the stored reference signature; and
generate the output signal based on the comparison;
wherein the reference signature and the measurement signature conform to a predetermined format.

16. The server of claim 15, wherein the predetermined format comprises a template for a plurality of characters arranged in a predetermined order.

17. The server of claim 15, wherein the output signal provides an indication of access permission.

18. The server of claim 15, further comprising an authentication unit.

19. The server of claim 15, wherein the memory is further configured to map a user with the reference signature.

20. A system, comprising:
an electronic device, having at least one sensor configured to detect at least one physical response;
a communication unit configured to transmit and receive data; and
a processor configured to:
control output of a stimulus to a subject;
generate a measurement signature based on the detected at least one physical response; and
generate an output signal;
wherein the measurement signature conforms to a predetermined format; and
the server of claim 15.

21. (canceled)
